(19) United States
(12) Patent Application Publication
(10) Pub. No.: US 2023/0355409 A1
(43) Pub. Date: Nov. 9, 2023

(54) ORTHOPAEDIC IMPLANT TOOL INTERFACING SOCKET

(71) Applicant: SIGNATURE ORTHOPAEDICS EUROPE LTD, Dublin (IE)

(72) Inventors: Declan BRAZIL, Lane Cove West (AU); Chris BURGESS, Dublin (IE); John MANFREDI, Dublin (IE)

(21) Appl. No.: 18/246,282

(22) PCT Filed: Oct. 8, 2021
(86) PCT No.: PCT/AU2021/051175

§ 371 Date: Mar. 22, 2023

(30) Foreign Application Priority Data
Oct. 8, 2020 (AU) 2020903641

(51) Int. Cl. A61F 2/46

(52) U.S. Cl. CPC A61F 2/4607; A61F 2002/4625; A61F 2002/4619

(57) ABSTRACT

An orthopaedic implant tool interfacing socket for an orthopaedic implant has a stage spaced from an entrance of a threaded blind hole thereunderneath. The stage at least partially defines an impaction face, a keyed entrance therethrough and an extraction tool engagement edge thereunderneath being out of rotational alignment with the keyed entrance.

### FIELD OF THE INVENTION

This invention relates generally to an orthopaedic implant tool interfacing socket.

### BACKGROUND OF THE INVENTION

Orthopaedic implants generally require a feature to allow a surgeon to manipulate the implant into position. For example, femoral prosthetic devices for hip arthroplasty are inserted into a patient's femur with an impacting tool interfacing the prosthetic device.

Some implants comprise an indentation which locates a tip of an insertion tool which may then be impacted to drive in the implant.

Other implants comprise a threaded socket into which an insertion tool is screwed which may also assist the extraction of the implant. For example, US 20180311050 A1 (DEPUY SYNTHES PRODUCTS, INC.) 1 Nov. 2018 discloses an implant having neck proximal end portion with an insertion/extraction cavity which engages a threaded shaft of an instrument for insertion or extraction.

However, threading may be damaged by inadvertent cross threading or by the insertion tool not being correctly seated during impaction.

Damaged threading may prevent or hinder disengagement of the insertion tool or, worse, removal of the implant as may be required during initial or revision surgery.

The present invention seeks to provide a way to overcome or substantially ameliorate at least some of the deficiencies of the prior art, or to at least provide an alternative.

It is to be understood that, if any prior art information is referred to herein, such reference does not constitute an admission that the information forms part of the common general knowledge in the art, in Australia or any other country.

### SUMMARY OF THE DISCLOSURE

There is provided herein an orthopaedic implant tool interfacing socket for an orthopaedic implant which has a stage spaced from an entrance of a threaded blind hole thereunderneath. The stage at least partially defines an impaction face, a keyed entrance therethrough and an extraction tool engagement edge thereunderneath being out of rotational alignment with the keyed entrance.

The impaction face is preferably indented and may be hemi-spheroidal.

The threading is protected deep within the socket which may avoid cross threading and/or incorrect tool seating.

An insertion tool having a rod having a widened portion at a distal end thereof may be used to impact the impaction surface from differing angles to, for example, manipulate the implant around corners as may sometimes be required for direct anterior approach insertion of hip implants.

The widened portion and the impaction surface may be hemi-spherical to equally allow for impaction from various angles or non-hemi-spherical such as being hemi-elliptical to bias the insertion tool within an impaction angle range.

An insertion tool further comprising a distal threaded rod tip extending distally from the widened portion may screw into the threading of the threaded blind hole wherein and the widened portion abuts against the impaction face to take a load off the threading.

Furthermore, a rotation tool comprising a key fitting and locating within the keyed entrance may be used to control the version of the implant.

Furthermore, an extraction tool comprising a key fitting through the keyed entrance may be used to engage the extraction tool engagement edge to quickly and easily remove the implant.

According to one aspect, there is provided apparatus comprising an orthopaedic implant comprising an orthopaedic implant tool interfacing socket, the socket having a stage spaced from an entrance of a threaded blind hole thereunderneath, the stage at least partially defining an impaction face, a keyed entrance therethrough and an extraction tool engagement edge thereunderneath being out of rotational alignment with the keyed entrance.

The impaction face may be indented. The impaction face may be hemi-spheroidal. Alternatively, the impaction face may be non-hemi-spheroidal, such as hemi-elliptical.

In cross-section perpendicular with a longitudinal axis of the blind hole, the keyed entrance may comprise a central section being larger in cross-section than that of the threaded blind hole. The central section may be circular in cross-section.

In cross-section perpendicular with a longitudinal axis of the blind hole, the keyed entrance may be elongate in cross-section wherein the keyed entrance may define a pair of slots diametrically opposite the central section.

The socket may define a frustoconical lead-in from beneath the stage into the threaded blind hole.

The apparatus may further comprise an insertion tool comprising a rod and a widened section at a distal end thereof and wherein the insertion tool engages the socket such that the widened section fits the impaction face thereof.

The insertion tool may comprise a threaded rod tip extending distally from the widened section which screws into the threaded blind hole. The threaded rod tip may screw into the threaded blind hole until the widened section abuts the impaction face.

The impaction face and the widened section may be hemi-spheroidal. Alternatively, the impaction face and the widened section are non-hemi-spheroidal such as hemi-elliptical.

The apparatus may further comprise an extraction tool comprising a rod and a key at a distal end thereof, the key shaped to fit through the keyed entrance and engage under the extraction tool engagement edge when rotated out of alignment with the keyed entrance.

The apparatus may further comprise a rotation tool comprising a rod and a key at a distal end thereof, the key shaped to fit into the keyed entrance and wherein the rotation tool has a feature which locates the key within the keyed entrance when the rotation tool may be pressed into the socket.

The feature may comprise a platform above the key which abuts against the impaction face.

There are also provided methods for inserting, extracting and rotating an implant having the socket with the various tools.

Other aspects of the invention are also disclosed.

### BRIEF DESCRIPTION OF THE DRAWINGS

Notwithstanding any other forms which may fall within the scope of the present invention, preferred embodiments of the disclosure will now be described, by way of example only, with reference to the accompanying drawings in which:

FIG. 1 shows a top perspective view of an orthopaedic implant comprising an orthopaedic implant tool interfacing socket in accordance with an embodiment;

FIG. 2 shows a top plan view of the implant;

FIG. 3 shows a cross-sectional side elevation view of the implant;

FIG. 4 shows a top perspective magnified view of the socket;

FIG. 5 shows a cross-sectional magnified elevation view of the socket;

FIG. 6 shows an insertion tool in accordance with an embodiment;

FIG. 7 shows a further insertion tool in accordance with an embodiment; and

FIG. 8 shows an extraction tool in accordance with an embodiment.

### DESCRIPTION OF EMBODIMENTS

FIGS. 1-3 show an example of an orthopaedic implant tool interfacing socket 100 applied to a hip implant 101. The implant 101 may comprise a femoral stem 102 and a neck 103 which may have a trunnion 104 which engages a femoral head which goes into a liner of an acetabular cup.

The socket 100 may however be applied to other types of orthopaedic implants requiring insertion and/or extraction.

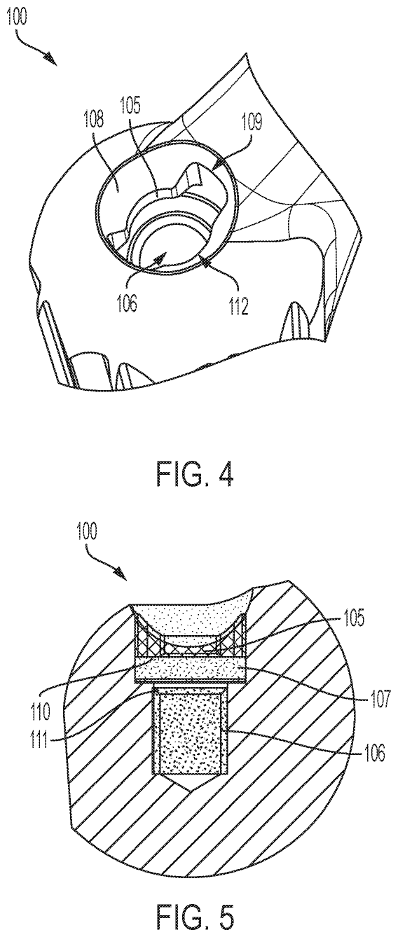

With reference to FIGS. 4 and 5, the socket 100 has a stage 105 spaced from and entrance of a threaded blind hole 106 thereunderneath.

The spacing of the stage 105 from the entrance of the blind hole 106 may form a void 107 therebetween. The socket 100 may comprise a slight frustoconical lead-in 111 from the void 107 into the threaded blind hole 106.

The stage 105 at least partially defines an impaction face 108 and a keyed entrance 109 therethrough. The stage 105 further defines an extraction tool engagement edge 110 thereunderneath being out of rotational alignment with the keyed entrance 109.

The impaction face 108 is preferably indented. The impaction face 108 may be hemi-spheroidal. In one embodiment, the impaction face 108 may be hemi-spherical (i.e., forming part of a sphere of constant radius). In alternative embodiments, the impaction face 108 may be non-hemi-spherical such as being hemi-elliptical.

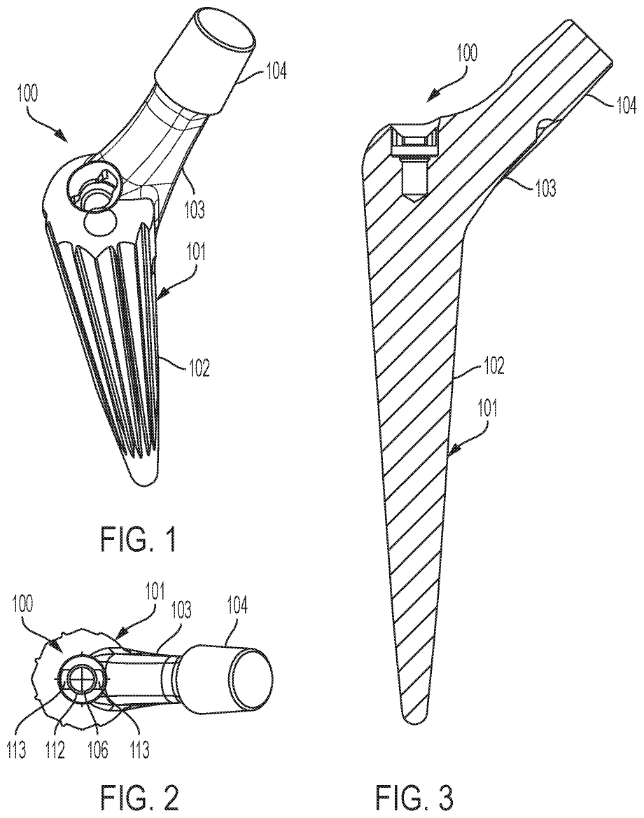

As is best shown in FIG. 2, in cross-section perpendicular with a longitudinal axis of the blind hole 106, the keyed entrance 109 may comprise a central section 112 being larger in cross-section than that of the threaded blind hole 106. The central section 112 may be circular. The keyed entrance 109 may be elongate and may define a pair of slots 113 diametrically opposite the central section 112.

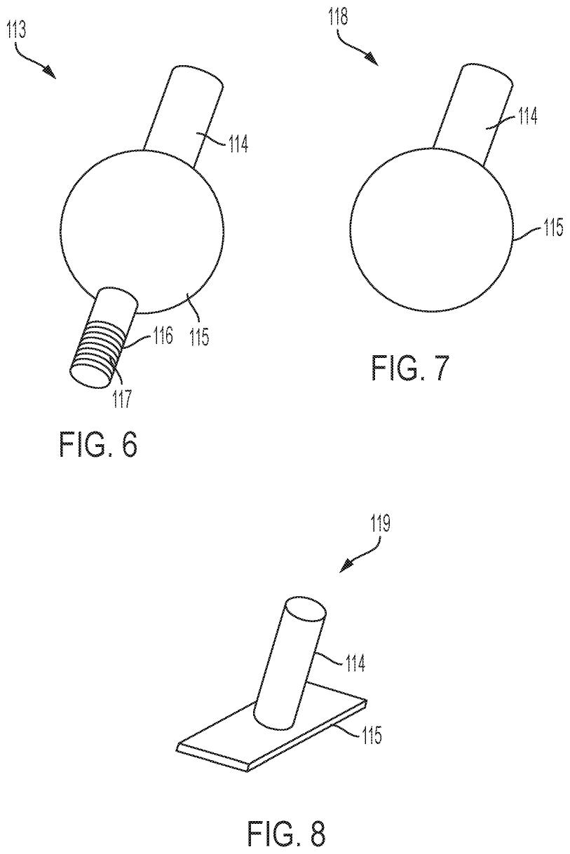

FIG. 6 shows an insertion tool 113 comprising a rod 114, a widened portion 115 and a rod tip 116. The widened portion 115 fits the impaction face 108. The rod tip 116 comprises threading 117 at the distal portion thereof. The rod tip 116 may be inserted through the entrance 109 and the rod 114 turned to screw the threading 117 thereof into the threading of the threaded blind hole 106 until the undersurface of the widened portion 115 contacts against the impaction face 108. The implant 101 may then be driven in by impacting the rod 114 wherein the undersurface of the widened portion 115 abutting the impaction face 108 reduces the force applied via the threading 106.

FIG. 7 shows an insertion tool 118 comprising the rod 114 and the widened portion 115 but which is devoid of the rod tip 116 which may be applied to impact the face 108 at various angles.

FIG. 8 shows an extraction tool 119 comprising a rod 114 comprising an elongate key 115 shaped to fit through the entrance 109 and which can be rotated out of alignment with the entrance 109 to engage under the extraction tool engagement edge 110 to pull out the implant 101. The extraction tool 119 may comprise a proximal slap handle (not shown).

In embodiments, a rotation tool (not shown) may be employed which comprises a key which is shaped to fit and locates within the shaped entrance 109. The rotation tool may comprise a feature which locates the key within the shaped entrance 109. For example, the rotation tool may comprise a platform above the key which abuts against the impaction face 108 to locate the key within the shaped entrance. As such, when the rotation tool is twisted, the shaped key thereof engages the keyed entrance 109 to rotate the implant.

The foregoing description, for purposes of explanation, used specific nomenclature to provide a thorough understanding of the invention. However, it will be apparent to one skilled in the art that specific details are not required in order to practise the invention. Thus, the foregoing descriptions of specific embodiments of the invention are presented for purposes of illustration and description. They are not intended to be exhaustive or to limit the invention to the precise forms disclosed as obviously many modifications and variations are possible in view of the above teachings. The embodiments were chosen and described in order to best explain the principles of the invention and its practical applications, thereby enabling others skilled in the art to best utilize the invention and various embodiments with various modifications as are suited to the particular use contemplated. It is intended that the following claims and their equivalents define the scope of the invention.

The term “approximately” or similar as used herein should be construed as being within 10% of the value stated unless otherwise indicated.

1. Apparatus comprising an orthopaedic implant comprising an orthopaedic implant tool interfacing socket, the socket having a stage spaced from an entrance of a threaded blind hole thereunderneath, the stage at least partially defining an impaction face, a keyed entrance therethrough and an extraction tool engagement edge thereunderneath being out of rotational alignment with the keyed entrance.

2. Apparatus as claimed in claim 1, wherein the impaction face is indented.

3. Apparatus as claimed in claim 2, wherein the impaction face is hemi-spheroidal.

4. Apparatus as claimed in claim 2, wherein the impaction face is non-hemi-spheroidal.

5. Apparatus as claimed in claim 4, wherein the impaction face is hemi-elliptical.

6. Apparatus as claimed in claim 1, wherein, in cross-section perpendicular with a longitudinal axis of the blind hole, the keyed entrance comprises a central section being larger in cross-section than that of the threaded blind hole.

7. Apparatus as claimed in claim 6, wherein the central section is circular in cross-section.

8. Apparatus as claimed in claim 6, wherein, in cross-section perpendicular with a longitudinal axis of the blind hole, the keyed entrance is elongate in cross-section.

9. Apparatus as claimed in claim 8, wherein the keyed entrance defines a pair of slots diametrically opposite the central section.

10. Apparatus as claimed in claim 1, wherein the socket defines a frustoconical lead-in from beneath the stage into the threaded blind hole.

11. Apparatus as claimed in claim 1, further comprising an insertion tool comprising a rod and a widened section at a distal end thereof and wherein the insertion tool engages the socket such that the widened section fits the impaction face thereof.

12. Apparatus as claimed in claim 11, wherein the insertion tool further comprises a threaded rod tip extending distally from the widened section which screws into the threaded blind hole.

13. Apparatus as claimed in claim 12, wherein the threaded rod tip screws into the threaded blind hole until the widened section abuts the impaction face.

14. Apparatus as claimed in claim 11, wherein the impaction face and the widened section are hemi-spheroidal.

15. Apparatus as claimed in claim 11, wherein the impaction face and the widened section are non-hemi-spheroidal.

16. Apparatus as claimed in claim 15, wherein the impaction face and the widened section are hemi-elliptical.

17. Apparatus as claimed in claim 1, further comprising an extraction tool comprising a rod and a key at a distal end thereof, the key shaped to fit through the keyed entrance and engage under the extraction tool engagement edge when rotated out of alignment with the keyed entrance.

18. Apparatus as claimed in claim 1, further comprising a rotation tool comprising a rod and a key at a distal end thereof, the key shaped to fit into the keyed entrance and wherein the rotation tool has a feature which locates the key within the keyed entrance when the rotation tool is pressed into the socket.

19. Apparatus as claimed in claim 18, wherein the feature comprises a platform above the key which abuts against the impaction face.

20. A method inserting the implant as claimed in claim 1 using an insertion tool as claimed in claim 11 or 12.

21. A method of extracting an implant as claimed in claim 1 using an extraction tool as claimed in claim 17.

22. A method of rotating an implant as claimed in claim 1, using a rotation tool as claimed in claim 18.
